(19) United States
(12) Patent Application Publication
(10) Pub. No.: US 2019/0022135 A1
(43) Pub. Date: Jan. 24, 2019

(54) TREATMENT FOR CHITIN-CONTAINING MICROORGANISMS

(71) Applicant: Benchmark Animal Health Limited, Sheffield (GB)

(72) Inventors: Shimaa E. ALI, Portland, ME (US); Even THOEN, Portland, ME (US); Ida SKAAR, Portland, ME (US); Øystein EVENSEN, Portland, ME (US)

(21) Appl. No.: 16/069,419

(22) PCT Filed: Jan. 12, 2016
(86) PCT No.: PCT/EP2016/050466

§ 371 Date: Jul. 11, 2018

(51) Int. Cl. A61K 33/22; A23K 50/80; A61P 33/02; A23K 20/10; A23K 20/20; A61P 31/10; A61P 33/14

(52) U.S. Cl. CPC A61K 33/22; A23K 50/80; A61P 33/02; A23K 20/10; A61K 31/19; A61P 31/10; A61P 33/14; A61K 2300/00; A23K 20/20

(57) ABSTRACT

The present disclosure relates to compositions for the treatment of chitin-containing microorganism infection or colonization of an animal. The methods, systems and kits provided herein facilitate termination and/or inhibition of proliferation of chitin-containing microorganisms (e.g., Saprolegnia and sea lice).

The invention relates to methods for treating chitin-containing microorganisms. Embodiments of the invention relate to methods for treating chitin-containing microorganism infection or colonisation, kits for treating chitin-containing microorganism infection or colonisation and generally to systems for farming aquatic animals that inhibit proliferation of chitin-containing microorganisms.

### BACKGROUND

In general, animals are exposed to environments with high microorganism populations. When microorganisms breach the physical and/or immune system of an animal, infectious diseases may result.

For example, aquatic animals (e.g., fish) are exposed to water with a high microorganism population. In the wild, the infectious diseases are normally at low levels because the water in which the animals live circulates freely. However, in a crowded and/or limited space (e.g. fish farms), such infection can become epidemic and the infectious diseases may be transferred to wild animal populations. In aquaculture industry, infectious diseases cause huge economic losses, for example, fish and ova.

There is a need in the art for rapid and effective treatment of a microorganism infection of individual animals and to prevent the spread of disease to healthy animals.

### SUMMARY OF THE INVENTION

According to the present invention, as seen from a first aspect, there is provided a composition for the treatment of chitin-containing microorganism infection or colonization of an animal, the composition comprising a mineral acid and a carboxylic acid.

The mineral acid may be any mineral acid suitable for the treatment of chitin-containing microorganism infection.

Preferably, the mineral acid is boric acid (BA) HBO₃, a functional equivalent or a functional derivative thereof.

The carboxylic acid may be any carboxylic acid suitable for the treatment of chitin-containing microorganism infection.

Preferably, the carboxylic acid is propionic acid (PA) CH₃CH₂COOH, a functional equivalent or a functional derivative thereof.

Preferably, the composition has an effective concentration of the mineral acid not less than about 0.2 units and an effective concentration of the carboxylic acid not less than about 0.2 units.

The composition may have an effective concentration of mineral acid between about 0.2 units and about 4 units. The composition may have an effective concentration of carboxylic acid between about 0.2 units and about 10 units.

In some embodiments of the invention, the effective concentration of mineral acid in the composition may be greater than about 0.2 units.

Embodiments of the invention may comprise an effective concentration of mineral acid between about 0.2 units and about 4 units, between about 1 unit and about 4 units, between about 0.2 units and about 1.4 units, between about 0.2 units and about 1 units, between about 0.2 units and about 0.5 units, between about 0.5 units and about 1 units or between about 0.1 units and about 0.2 units.

Preferably, the effective concentration of carboxylic acid may be greater than about 0.2 units. Embodiments of the invention may comprise an effective concentration of carboxylic acid between about 0.2 units and about 10 units, between about 0.2 units and about 1.0 units or between about 2 units and about 10 units.

The units may be g/L for example where the composition is a liquid composition or g/dm³ for example where the composition is a solid composition.

Preferably, the composition may comprise one or more stabilisers, additives or excipients.

In some embodiments of the present invention, the animal to be treated for chitin-containing microorganism infection or colonization is an aquatic animal.

The aquatic animal may be a fish selected from the group consisting of: a fish egg, a Juvenile fish, a fry, a fingerling, an adult fish, and an off-spring of the fish.

The aquatic animal may be a fish selected from the group consisting of: a brown trout, an Atlantic salmon, a rainbow trout, a coho salmon, a catfish e.g. channel catfish, a pike, an arctic char, an eel, a roach, a carp, a sturgeon, a kissing gourami, a guppy, a swordfish, a tilapia, a cod and a platyfish.

In alternative embodiments, the aquatic animal may be selected from the group consisting: an aquatic mammal, an aquatic bird, an aquatic reptile, an amphibian, and an aquatic invertebrate.

Preferably, the animal has the chitin-containing microorganism infection or colonization. The chitin-containing microorganism may comprise an oomycete which may comprise at least one of a Saprolegnia, an Aphanomyces, or a Branchiomyces.

In some embodiments, the chitin-containing microorganism comprises a Saprolegnia.

The chitin-containing microorganism infection may be associated with a sea louse. The sea louse may be a sea louse of at least one of a genera: Lepeophtheirus, Caligus, Caligus rogercresseyi, Caligus clemensi, Caligus chiastos, Caligus epidemicus, Caligus elongates, or Lepeophtheirus salmonis.

According to the present invention, as seen from a second aspect, there is provided a composition as herein described for use as a medicament for an animal. Preferably, the animal is an aquatic animal.

According to the present invention, as seen from a third aspect, there is provided a composition as herein described for use in the treatment of chitin-containing microorganism infection or colonisation.

According to the present invention, as seen from a fourth aspect, there is provided a method for treating chitin-containing microorganism infection or colonization, the method comprising:
providing a composition as herein described comprising a mineral acid and a carboxylic acid; and
contacting an animal with the composition.

The effective concentration of mineral acid in the composition may be greater than about 0.2 units. Embodiments of the invention may comprise an effective concentration of mineral acid between about 0.2 units and about 4 units, between about 1 unit and about 4 units, between about 0.2 units and about 1.4 units, between about 0.2 units and about 1 units, between about 0.2 units and about 0.5 units, between about 0.5 units and about 1 units or between about 0.1 units and about 0.2 units.

The effective concentration of carboxylic acid may be greater than about 0.2 units. Embodiments of the invention may comprise an effective concentration of carboxylic acid between about 0.2 units and about 10 units, between about 0.2 units and about 1.0 units or between about 2 units and about 10 units.

The units may be g/L for example where the composition is a liquid composition or g/dm³ for example where the composition is a solid composition.

In some embodiments, the method further comprises the step of dissolving the composition in a carrier to provide an anti-microorganism solution and wherein the contacting step comprises contacting the animal with the anti-microorganism solution. Preferably, the carrier comprises water. In this embodiment, it will be understood by the skilled person that the effective concentration of mineral acid and carboxylic acid refers to the concentrations in the anti-microorganism solution and the units will be g/L.

In the step of contacting the animal, the mineral acid and the carboxylic acid may contacted with the animal sequentially, concomitantly or simultaneous. Alternatively, any other suitable regime for contacting the animal with the mineral acid and the carboxylic acid may be employed.

Preferably, the animal is contacted with the composition for a period of time of between about 24 hours and about 96 hours. Preferably, the animal is an aquatic animal.

According to the present invention, as seen from a fifth aspect, there is provided a kit for administering a composition as described herein to an animal to treat chitin-containing microorganism infection or colonization.

Preferably, the kit comprises: a container having the composition, the composition dissolvable in a carrier to provide an anti-microorganism solution; and packaging material that includes an instruction directing contacting an animal with the anti-microorganism solution to treat chitin-containing microorganism infection or colonization.

The kit may direct that the mineral acid and the carboxylic acid are contacted with the animal sequentially, concomitantly or simultaneous. Alternatively, any other suitable regime for contacting the animal with the mineral acid and the carboxylic acid may be directed.

Preferably, the kit directs that the animal is contacted with the composition for a period of time of between about 24 hours and about 96 hours. Preferably, the animal is an aquatic animal.

According to the present invention, as seen from a sixth aspect, there is provided a system for farming an animal, comprising: a farming system for farming the animal, the farming system including marine or fresh water containing the composition described herein in a sufficient quality to inhibit proliferation of chitin-containing microorganisms.

The system may comprise contacting the mineral acid and the carboxylic acid with the animal sequentially, concomitantly or simultaneous. Alternatively, any other suitable regime for contacting the animal with the mineral acid and the carboxylic acid may be employed in the system.

Preferably, the animal is contacted with the composition for a period of time of between about 24 hours and about 96 hours. Preferably, the animal is an aquatic animal.

According to the present invention, as seen from a seventh aspect, there is provided an animal treated with the composition described herein. Preferably, the animal is an aquatic animal, preferably an aquatic animal described herein.

### DESCRIPTION OF THE DRAWINGS

FIG. 1 illustrates the viability of saprolegnia spores after exposure to different treatment concentrations of boric acid (BA), propionic acid (PA) and pyzece after 4 hours.

FIG. 2 illustrates the reduction of viability relative to non-treated control corresponding to the data in FIG. 1.

### DETAILED DESCRIPTION

The present invention provides a composition for the treatment of chitin-containing microorganism infection or colonization of an animal, the composition comprising a mineral acid and a carboxylic acid.

Preferably the mineral acid is boric acid (BA). Preferably, the carboxylic acid is propionic acid (PA).

Those skilled in the art should be familiar with the compounds ‘boric acid’ and ‘propionic acid’.

The term “boric acid” (BA) refers to an organic compound with the formula H₃BO₃ (sometimes written B(OH)₃) or any chemical compound containing parts or traces of boric acid H₃BO₃.

Boric acid is a weak, monobasic Lewis acid of boron. Derivatives of boric acid comprising boron oxyanions (e.g. salts and esters of boric acid) are known as borates. The mineral acid may be functional equivalent of boric acid or a functional derivative of boric acid e.g. a borate.

The term “propionic acid” (PA) refers to an organic compound with the formula CH₃CH₂COOH, or any chemical compound containing parts or traces of propionic acid CH₃CH₂COOH.

Propionic acid is a carboxylic acid. The anion, salts and esters of propionic acid are known as propionates (propanoates). The carboxylic acid may be a functional equivalent of propionic acid or a functional derivative of propionic acid e.g. a propionate.

The amount of BA and PA used for treatment should be present in a sufficient quantity to inhibit proliferation of chitin-containing microorganisms.

Preferably, the composition has an effective concentration of the mineral acid not less than about 0.2 units and the concentration of the carboxylic acid is not less than about 0.2 units.

By effective concentration, it is meant the average concentration of the component e.g. mineral acid that comes into contact with the animal. The effective concentration is the concentration that is sufficient to induce a response in the pathogen/parasite (chitin-containing microorganism).

The composition may have an effective concentration of the mineral acid between about 0.2 units and about 4 units. The composition may have an effective concentration of the carboxylic acid between about 0.2 units and about 10 units.

The units may be g/L for a liquid composition or g/dm³ for a solid composition.

For example, the concentration of mineral acid may be no less than about 0.2 g/L and the concentration of carboxylic acid no less than about 0.2 g/L in a liquid composition. The concentration of mineral acid and carboxylic acid may be no less than 0.2 g/dm³ in a solid composition.

The terms ‘liquid composition’ and ‘solid composition’ will be readily understood by those skilled in the art. For example, a liquid composition is a composition that is substantially liquid and concentrations may be referred to in e.g., g/L or g/dm³. A solid composition is a composition that is substantially solid and concentrations may be referred to in g/dm³.

In some embodiments where the composition is a liquid composition, the concentration of the mineral acid may be greater than about 0.2 g/L. Other embodiments of the invention may comprise a concentration of mineral acid between about 0.2 g/L and about 4 g/L, between about 1 g/L and about 4 g/L between about 0.2 g/L and about 1.4 g/L, between about 0.2 g/L and about 1 g/L, between about 0.2 g/L and about 0.5 g/L, between about 0.5 g/L and about 1 g/L or between about 0.1 g/L and about 0.2 g/L.

In some embodiments where the composition is a liquid composition, the concentration of the carboxylic acid may be greater than about 0.2 g/L. Other embodiments may comprise a concentration of carboxylic acid between about 0.2 g/L to about 10 g/L, between about 0.2 to about 1.0 g/L or between about 2 g/L to about 10 g/L.

The concentration of carboxylic acid may range from about 31.4 mM to about 125.6 mM.

In certain embodiments, the composition may be dissolved in a carrier to provide an anti-microorganism solution prior to treatment of an animal. In these embodiments, it will be understood that the effective concentrations refer to the concentration of the mineral acid and carboxylic acid in the anti-microorganism solution.

In some embodiments, for example for treatment of terrestrial animals, it is envisaged that the composition may be in provided as a solid (dry) composition, for example, in pulverised form. The solid composition may be in a form suitable for spraying.

It will be appreciated by the skilled person that the effective concentration ranges described herein may vary depending on circumstances, for example, the severity of the infection or colonisation, the type of chitin-containing microorganism, the animal that is to be treated and the mode of treatment e.g. if a carrier is employed.

Preferably, the composition may comprise one or more stabilisers, additives or excipients. Examples of possible stabilisers include but are not limited to alginate, chitosan, or gelatin. Examples of possible additives include but are not limited to span, tween or other emulsifiers/stabilisers. Examples of possible excipients include but are not limited to sucrose, lactose or polysaccharides.

The present invention also provides a composition described herein for use as a medicament for an animal. Preferably, the animal is an aquatic animal.

The present invention also provides a composition described herein for use in the treatment of chitin-containing microorganism infection or colonisation.

According to the present invention, there is provided a method for treating chitin-containing microorganism infection or colonization. The method may comprise the steps of:
providing a composition as herein described comprising a mineral acid and a carboxylic acid; and
contacting an animal with the composition.

There is also provided a method for administering an anti-microorganism composition to an animal to treat chitin-containing microorganism infection or colonization.

In some embodiments, the method may comprise the steps:
dissolving an anti-microorganism composition as herein described in a carrier to provide an anti-microorganism solution; and
contacting an animal with the anti-microorganism solution.

Preferably, the carrier comprises water, for example, the carrier may be comprised substantially of marine water or fresh water.

In some embodiments, the method may comprise the step:
contacting the animal with marine or fresh water (carrier) containing a mineral acid and a carboxylic acid in a sufficient quantity to inhibit proliferation of the chitin-containing microorganism.

The method may further comprise the step of removing the aquatic animal from the anti-microorganism solution.

Preferably, the mineral acid is boric acid (BA), a functional equivalent or functional derivative thereof. Preferably, the carboxylic acid is propionic acid (PA), a functional equivalent or a functional derivative thereof. Preferably, the animal is an aquatic animal.

According to the present invention, there is also provided a kit for administering an anti-microorganism composition to an aquatic animal to treat chitin-containing microorganism infection or colonization, the kit comprising:

a container having the anti-microorganism composition described herein comprising a mineral acid and a carboxylic acid, the anti-microorganism composition dissolvable in a carrier to provide an anti-microorganism solution; and packaging material that includes an instruction directing contacting the animal with the anti-microorganism composition to treat the chitin-containing microorganism infection or colonization.

Alternatively, the instruction may direct administration of the anti-microorganism composition to the animal to treat the chitin-containing microorganism infection or colonisation.

According to the present invention, there is also provided a system for farming an animal, comprising:
a farming system for farming the animal, the farming system including marine or fresh water containing the composition described herein, including a mineral acid and a carboxylic acid, in a sufficient quantity to inhibit proliferation of chitin-containing microorganisms.

Preferably, the animal is an aquatic animal. The carrier may be any suitable carrier or combination of carriers. Preferably the carrier is water or consists substantially of water. Other compounds may also be dissolved in the carrier e.g., sucrose, lactose or polysaccharides.

The mineral acid and the carboxylic acid may be dissolved in the carrier to provide an anti-microoganism solution. The mineral acid and carboxylic acid may combined prior to the dissolving step or may remain separate prior and be dissolved simultaneously or sequentially in the carrier.

It would be appreciated by the skilled person that the animal may be contacted with the carrier prior to addition of the mineral acid and/or carboxylic acid or after addition of the mineral acid and/or carboxylic acid.

In some embodiments, the mineral acid and carboxylic acid may be added concomitantly to the carrier to form the anti-microorganism solution. Alternatively, the mineral acid and carboxylic acid may be added sequentially in any order depending on the treatment regimen. Alternatively, the mineral acid and carboxylic acid may be added simultaneously to the carrier.

For example, the mineral acid may be dissolved in the carrier to provide an anti-microorganism solution and the animal may be contacted before or after a subsequent dissolution of the carboxylic acid in the carrier or vice versa.

Additional variations and modifications of the treatment regimen will be appreciated by those skilled in the art.

In some embodiments, an animal may be contacted (or directed to be contacted) with the anti-microorganism solution for a period of time of between about 24 hours and about 96 hours.

Preferably, the animal may be contacted with the anti-microorganism solution for a predetermined number of times within 24 hours. For example, the animal may be contacted with the anti-microorganism solution for at least one of 1, 2, 3, 4, 5, 6, or 8 hours.

The following features may relate to any aspect of the present invention (composition, medical use, method of treatment, kit and system).

Preferably, the aquatic animal is selected from the group consisting of a fish, an aquatic mammal, an aquatic bird, an aquatic reptile, an amphibian, and an aquatic invertebrate. Preferably, the aquatic animal is a fish.

In some embodiments, the fish is a farmed fish. The fish may comprise at least one selected from the group: a brown trout, an Atlantic salmon, a rainbow trout, a coho salmon, a catfish e.g. channel catfish, a pike, an arctic char, an eel, a roach, a carp, a sturgeon, a kissing gourami, a guppy, a swordfish, or a platyfish.

In some embodiments, the fish may comprise at least one selected from the group: a fish egg, a Juvenile fish, a fry, a fingerling, an adult fish or an off-spring of the fish.

Preferably, the aquatic animal has the chitin-containing microorganism infection or colonization.

The chitin-containing microorganism infection may be associated with an oomycete including at least one of a Saprolegnia, an Aphanomyces, or a Branchiomyces.

The chitin-containing microorganism infection may be associated with a Saprolegnia.

The chitin-containing microorganism infection may be associated with an ectoparasite. The ectoparasite may comprise a sea louse wherein the sea louse is at least one genera selected from the group Lepeophtheirus, Caligus, Caligus rogercresseyi, Caligus clemensi, Caligus chiastos, Caligus epidemicus, Caligus elongates, or Lepeophtheirus salmonis.

### Microorganisms

Chitin (C₈H₁₃O₅N)ₙ is a long-chain polymer of a N-acetylglucosamine, derived from glucose. Chitin is the main component of the cell walls of fungi and oomycetes (e.g., Saprolegnia sp.) that infect freshwater fish, the exoskeletons of arthropods (e.g., crustaceans and copepods). For example, Caligidae parasites of fish (i.e., external parasites) can be found on mucus and skin of wild and farmed fish. The structure of chitin is comparable to the polysaccharide cellulose and forms crystalline nanofibrils. By function it may be compared to the protein keratin.

Chitin is the building block of exoskeletons that is the external skeleton that supports and protects the body of animals such as grasshoppers and cockroaches, crustaceans (e.g., crabs and lobsters), and arthropods (e.g., copepods). For example, arthropods may include Caligidae parasites of fish (e.g., sea louse (Lepeophtheirus salmonis) of salmonids and Caligus sp. of fish).

Exoskeletons contain rigid and resistant components that fulfil a set of functional roles including protection, excretion, sensing, support, feeding and acting as a barrier against desiccation in terrestrial organisms. Exoskeletons have a role in defence from pests and predators, support, and in providing an attachment framework for musculature. In addition to chitin, exoskeletons also contain calcium carbonate which makes them harder and stronger.

Saprolegnia spp. are generally termed “watermolds” and share common features with fungi and algae. The term saprolegniasis refers to any disease of fishes or fish eggs caused by species of the family Saprolegniaceae (Oomycotina). Symbiotic associations between fish and Saprolegnia spp. have been known for decades and the first description dates back to 1748 where saprolegniasis was reported in roach (Rutilus rutilus L.) in England. Since then saprolegniasis has been detected in a growing number of species in or on various fishes from all over the world. Particular interest has been paid from countries and regions with growing aquaculture industry since saprolegniasis causes high economic losses in fish and ova.

Saprolegnia infection is traditionally known as “fungal infection” in fish, and is typical seen in the freshwater stage of salmonids. Saprolegnia infections are visible to the naked eye as white patches on the skin of the fish or as “cotton wool” on fish eggs. The “fungal” patches may consist of one or more species of Saprolegnia and may become grey due to the presence of bacteria and debris. The disease was previously controlled by the use of malachite green, an organic dye which has proved very efficient in controlling all infectious stages of Saprolegnia spp. The use of malachite green has been banned in Norway and other parts of the world due to its potential toxicological effects. This has increased the incidence of Saprolegnia infections in the aquaculture all over the world, resulting in huge economic losses.

Saprolegnia spp. belong to the class Oomycetes, which is a group of fungi-like pathogens in the kingdom Straminiphila. Oomycetes have their phylogenetic roots with the chromophyte algae (which includes the diatoms, chrysophytes and brown seaweeds) rather than with the main evolutionary line of chitin containing fungi.

The Oomycetes are subdivided in orders and comprises several pests, like Phytophthora infestans causing the potato late blight, Aphanomyces astasi causing crayfish plaque, several fish pathogens from the genera Aphanomyces, Achlya and Saprolegnia and at least one species (Pythium insidiosum) with the potential of infecting humans and other mammals. In contrast to true fungi, Oomycetes contain little chitin in their walls, which instead is composed mainly of β-1-3-glucans.

Taxonomic classification of Saprolegnia is shown in Table 1.

TABLE 1
Taxonomic classification of Saprolegnia

| | |
|---|---|
| Kingdom | Straminiphila |
| Division | Oomycota |
| Class | Oomycetes |
| Order | Saprolegniales |
| Family | Saprolegniaceae |
| Genus | Saprolegnia |
| Species | 20-22 species of Saprolegnia |

Saprolegnia spp. are characterized by the growth of delicate, nonseptate hyphae and asexual reproduction by secondary zoospore discharge one by one in rapid succession through one exit pore in the sporangium. There are 20-22 species of Saprolegnia, most of them saphrophytic. Of these, 8-10 individual species have been implicated as causing saprolegniasis of salmonid fishes (Hughes, 1994), including S. australis, S. delica, S. diclina, S. ferax, S. monoica, S. parasitca, S. salmonis, S. shikotsuensis and S. tortulosa.

The most frequently examined and discussed Saprolegnia species are the fish pathogenic Saprolegnia parasitica and the less pathogenic species Saprolegnia diclina. Traditionally, Saprolegnia species are separated by the presence of sexual reproduction and characteristics of the gonads, i.e. the oogonia and the antheridia. However, the taxonomy is complex, and in particular the so-called “Saprolegnia parasitica-diclina complex” has led to a lot of confusion.

Saprolegnia species that grow on fish as parasites do not normally produce sexual structures during laboratory conditions. Absence of sexual reproductive structures is one of the primary distinguishing characteristics of Saprolegnia parasitica. The concept is that any Saprolegnia growing on a living fish, and not producing sexual reproductive structures, is, by definition, Saprolegnia parasitica. Saprolegnia species isolated from fish and with clusters of longspines in the secondary zoospore should be termed Saprolegnia parasitica as a practical approach.

Saprolegnia is homothallic, meaning that one single individual contains both male and female sex organs. The male and the female sex organs, the antheridium and the oogonium, respectively, are developed from the hyphae. Meiosis occurs to produce male nuclei and female eggs. The antheridia grow toward the oogonia and produce fertilization tubes that penetrate the oogonia.

Fertilization occurs when the male nuclei travel down these tubes to the female eggs and fuse with the female nuclei. This produces several thick-walled zygotes, called oospores. The number of oospores per oogonium is not constant, ranging from one to four in some species, to over fourty in others. Each oospore germinates into a new hypha which will produce a zoosporangium. From the zoosporangium the asexual reproduction, which is the main type of reproduction, occurs.

The pyriform primary zoospores, which are released from the zoosporangia are weak swimmers and function simply to disperse the spores from the immediate vicinity of the sporangium and parent colony. These primary zoospores settle down to produce 5-10 μm thin-walled cysts. This primary cyst acts as a miniature sporangium and releases a reniform secondary zoospore, which is the main motile stage. The secondary zoospore can maintain motility for many hours, even days, until it also encysts to produce a secondary cyst (syn. “encysted zoospore”, “zoospore cyst” or “cystospore”).

Then, they usually germinate into a new mycelium, on which sexual reproduction occurs, thus starting the reproduction cycle anew. The secondary cysts can also release new secondary-like zoospores which are able to encyst again. These repeated cycles of zoospore encystment and release of respectively secondary zoospores and cysts are called polyplanetism or repeated zoospore emergence.

Polyplanetism contributes to the fungus' pathogenicity by helping it to make several attempts locating a suitable culture medium to live on before settling down for good. After they have encysted the secondary zoospores develop hairs for attachment. It has been suggested that these hairs are also used for buoyancy to decrease sedimentation rate and for fungal-host recognition response and are important for pathogenesis.

Saprolegnia spp. are distributed worldwide in rivers and freshwater reservoirs. Over the last few decades, saprolegniasis have been reported frequently from all continents. International transfer of fish and eggs as part of the aquaculture industry is a possible risk factor when it comes to spread of different Saprolegnia species and strains throughout the world.

Saprolegnia infection is the single largest cause of economic losses in aquaculture and worldwide this disease is second only to bacterial diseases in economic importance. Fifty percent per year losses have been reported in elver (Angullia anguilla) and in coho salmon (Oncorhynchus kisutch) culture in Japan. In the south-eastern United States, major financial losses occur in channel catfish farming due to a condition called “winter kill” caused by Saprolegnia infections.

On infected fish, Saprolegnia parasitica form cotton-wool-like tufts on the integument. The early lesions are grey-white in colour and normally appear as circular colonies. The lesions are not randomly located, as head, fins and gills are more susceptible to infection. However, moribund fish may in severe cases have as much as 80% of the body surface area covered by Saprolegnia. Virulent strains of Saprolegnia spp. can cause very high mortalities (up to 100%) in many different salmonid species, and hyphae from Saprolegnia spp. are usually restricted to the integument and superficial musculature.

Histopathological changes beneath the superficial mycelia mat include dermal necrosis and oedema during the early stage, and deeper myofibrillar necrosis and extensive haemorrhage in the more progressive lesions. The tissue damage is probably caused by extracellular enzymes secreted by the advancing hyphae including hyphae penetration of the basement membrane. To date, there is no evidence to indicate that any Saprolegnia species produces toxins which could cause cellular damage at sites remote from the sites of hyphal invasion.

It is generally accepted that the ultimate cause of death is the severe haemodilution caused by haemorrhage and by the destruction of the waterproof properties of the fish integument. Typically, fish with severe saprolegniasis appear lethargic, lose the ability to position in the water column and generally do not recover and eventually perish. Saprolegnia infections of salmonid eggs are primarily associated with the aquaculture industry where large numbers of eggs, often of variable quality and viability, are incubated at relatively high densities.

Any dead eggs which are not removed from the incubation system are readily colonized by Saprolegnia, although this probably represents saprophytic colonisation by a range of fungi from the family Saprolegniaceae.

Saprolegnia spp. may infect both fish and ova of all types of salmonids. The economic importance of brown trout (Salmo trutta), Atlantic salmon (Salmo salar L.), rainbow trout (Oncorhynchus mykiss (Walbaum)) and coho salmon (Oncorhynchus kisutch) is the main reason that saprolegniasis has been subject to such strong focus in these species. However, Saprolegnia spp. can also infect a number of other teleost species. Catfish such as channel catfish (Ictalurus punctatus), pike (Esox lucius), arctic char (Salvelinus alpinus) and eel (Anguilla anguilla) as well as roach (Rutilus rutilus), carp (Cyprinidae spp.) and sturgeon (Acipenseridae spp.) have been infected with Saprolegnia. It has also been associated with tropical fish, including the kissing gourami (Helostoma temminckii), guppy (Poecilia reticulata), swordfish (Xiphias gladius) and platyfish (Xiphophorus couchianus).

The sea louse (plural sea lice) is a copepod within the order Siphonostomatoida, family Caligidae. There are more than 550 species in 37 genera, including approximately 162 Lepeophtheirus and 268 Caligus species. Sea lice are marine ectoparasites (external parasites) that feed on the mucus, epidermal tissue, and blood of host marine fish. The genera Lepeophtheirus and Caligus parasitize marine fish, in particular those species that have been recorded on farmed salmon.

Lepeophtheirus salmonis and various Caligus species are adapted to saltwater and are major ectoparasites of farmed and wild Atlantic salmon. Several antiparasitic drugs have been developed for control purposes. L. salmonis is the major sea louse of concern and has the most known about its biology and interactions with its salmon host. Caligus rogercresseyi has become a major parasite of concern on salmon farms in Chile. Recent evidence is also emerging that L. salmonis in the Atlantic Ocean has sufficient genetic differences from L. salmonis from the Pacific, showing that Atlantic and Pacific L. salmonis may have independently co-evolved with Atlantic and Pacific salmonids.

General understanding of the life-cycle and general biology of sea lice is for the main part based on laboratory studies designed to understand issues associated with sea lice infecting fish on salmon farms. Information on sea lice biology and interactions with wild fish is unfortunately sparse in most areas with a long-term history of open net-cage development, since understanding background levels of sea lice and transfer mechanisms have rarely been a condition of tenure license for farm operators.

Many sea louse species are species specific, for example L. salmonis has high specificity for salmonids, like farmed Atlantic salmon (Salmo salar). Lepeophtheirus salmonis can to some degree parasitize other salmonids, like brown trout (sea trout, Salmo trutta), arctic char (Salvelinus alpinus), and Pacific salmon species. Pacific L. salmonis can also develop on three-spined stickleback (Gasterosteus aculeatus) the life-cycle will not be completed. Temperature, light and currents depend the survival of sea lice (at different stages). Sea lice cannot live in freshwater and die and fall off anadromous fish such as salmonids as they return to freshwater. Atlantic salmon migrate to and swim upstream in the fall to reproduce, while the smolts return to saltwater the second spring to continue their life until adulthood.

Lepeophtheirus salmonis is approximately twice the size of most Caligus spp. (e.g. C. elongatus, C. clemensi, etc.). The body of sea lice consists of 4 regions: cephalothorax, fourth leg-bearing segment, genital complex, and abdomen. All species of lice have mouth parts shaped as a siphon or oral cone. The second antennae and oral appendages are modified to hold the parasite on the fish (attached stages). The adult females are significantly larger than males and develop a large genital complex which makes up the majority of the body mass. Two egg strings of 500 to 1000 eggs that get darker as they mature are approximately the same length as the female's body.

Sea lice have both free swimming (planktonic) and parasitic life stages. All stages are separated by moults. Eggs hatch into nauplius I which moult to a second naupliar stage and these stages are non-feeding stages. The copepodids are the infectious stage and search for an appropriate host. Currents, salinity, light, and other factors will assist copepodids in finding a host and settlement on the fish occurs in areas with the least hydrodynamic disturbance, typically fins and other protected areas. Attached copepodids will be attached to a suitable host for a period of time before moulting is induced, to chalimus I stage. Sea lice continue their development through 2 chalimus stages separated by a moult. They are attached to the host during this period. The pre-adult and adult stages are mobile (on the fish) and can also move between host fish.

Sea lice cause physical and enzymatic damage at attachment sites resulting in abrasion-like lesions that vary in severity depending and size. It is not clear whether stressed fish are particularly prone to infestation. Sea lice infection causes a generalized chronic stress response in fish. This can decrease the immune responses and render fish more susceptible to other diseases. Infection also impacts growth. The degree of damage is also dependent on the species of sea lice, the developmental stages that are present, and the number of sea lice on a fish.

Control of sea lice typically constitutes integrated pest management programs and these are recommended in Canada, Norway, Scotland, and Ireland, and also considered and partly implemented in Chile. Treatment of sea lice today includes chemical treatment with various compounds delivered in the water or via feed, cleaner fish and also other nonchemical treatment approaches (laser beads etc.).

The composition according to the present invention has been found to have use in the treatment of chitin-containing microorganism infection or colonisation of animals.

Surprisingly, it has been found that treatment with a combination of boric acid (BA) as the mineral acid and propionic acid (PA) as the carboxylic acid can inhibit germination and growth of Saprolegnia and decrease survival rates of sea lice.

Treatment with a combination of BA and PA was found to give the lowest viability scores for chitin-containing microorganisms when treating aquatic, without any associated toxicity to the aquatic animal within ranges given.

For example, no mortality was seen in Atlantic salmon (Salmo salar L.), 4-5 g in size when treated with a concentrations of BA up to 9 mg/L in combination with concentrations of PA up to 251.2 mM. Contrary to expectation, the concentrations of BA and PA described herein were found to be tolerable and safe for use for parr of Atlantic salmon, 4-5 g size.

Specifically, it has been found that boric acid interferes with the production and subsequent release of zoospores and vacuolation of the entire of zoosporangia.

It has been found that the presence of propionic acid has a direct toxic effect on spores and hyphae of Saprolegnia sp.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The term “aquatic animal” refers to any animal that spends all or some of the life in marine or fresh water. An “aquatic animal” can be, but is not limited to, a mammal such as a seal, sea lion, walrus, manatee, dugong, porpoise, dolphin, cetaceous or non-cetaceous whale, otter, or beaver; a bird such as, but not limited to, a web-footed bird such as a duck, goose, swan, gull, cormorant, penguin, a wading bird such as a coot, moor hen, flamingo, stork, heron; an aquatic reptile such as, but not limited to, an alligator, cayman, crocodile, turtle, snake or lizard; an amphibian such as, but not limited to, frogs, toads, newts and salamanders, neotenous larva or larvae thereof; fish and aquatic invertebrates such as, but not limited to, crustacea, insects, or molluscs.

The term “carrier” as used herein refers to any pharmaceutically acceptable solvent of antibiotics, chelating agents and pH buffering agents that will allow the antimicrobial composition of the present disclosure to be administered directly to an aquatic animal. A “carrier” as used herein, therefore, refers to such solvent as, but is not limited to, water, saline, physiological saline, ointments, creams, oil-water emulsions or any other solvent or combination of solvents and compounds known to one of skill in the art that is pharmaceutically and physiologically acceptable to the recipient animal. The term “carrier” further includes vitamin E or the like that may comprise an oily film over the site of application on the surface of an animal.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “derivative”, in the context of a chemical compound, it is meant a compound that has been derived from a similar compound by a chemical or physical process, for example by reaction such as by conjugation or by complexing with other chemical moieties and/or structures. As an example, borates can be considered derivatives of boric acid.

The term “fish” refers to any marine or freshwater fish species maintained in a tank, aquarium, pool, pond, aquaculture facility, fish farm, or any means other than the natural environment of the fish species. The term “fish” also refers to species and individuals thereof captured, rescued or taken from their native habitat and which may require treatment for microbial infestations. Fish species to which the methods of the present disclosure may be applied include, but are not limited to, ornamental fish, zebrafish, goldfish, koi, oscar, cichlids, tropical fish and fish for human or animal food such as, but not limited to, catfish, and salmonids such as trout, or salmon. In addition, examples of fish may include a brown trout, an Atlantic salmon, a rainbow trout, a coho salmon, a channel catfish, a pike, an arctic char, an eel, a roach, a carp, a sturgeon, a kissing gourami, a guppy, a swordfish, or a platyfish. Fish also refers to a fish of different stages between birth and adulthood, for example, eggs, juvenile fish, growing fish or a mature fish.

The term “disease” as used herein refers to a pathological condition recognizable as an abnormal condition of an animal. A “fish disease” is a pathological condition of fish that may be fatal or benign such as, but not limited to, ulcers, fin rot, dropsy, Malawi bloat disease, gill disease and columnaris, Saprolegnia infections, see louse infections, or saddlepatch disease.

As used herein, the terms “function” and “functional” and the like refer to a biological, enzymatic, or therapeutic function.

The terms “functional derivative” and “functional equivalent” in the context of a chemical compound refers to derivatives or equivalents that are structurally different but perform the same or similar function. Examples of functional derivatives of mineral acid include, e.g. salts and esters of boric acid, which are known as borates. The mineral acid may be functional equivalent of boric acid or a functional derivative of boric acid e.g. a borate. Examples of functional derivatives of carboxylic acid include anion, salts and esters of propionic acid which are known as propionates (propanoates). The carboxylic acid may be a functional equivalent of propionic acid or a functional derivative of propionic acid e.g. a propionate.

The terms “modulating” and “altering” include “increasing” and “enhancing” as well as “decreasing,” “inhibiting,” or “reducing”, typically in a statistically significant or a physiologically significant amount or degree relative to a control. In specific embodiments, anti-viral response level in response to viral infection is decreased relative to an unmodified or differently modified cell (i.e. a control) by at least 10%, at least 15%, at least 20%, at least 25%, at least 30%, at least 40%, at least 50%, at least 60%, at least 70%, at least 80%, at least 90%, at least 100%, at least 150%, at least 200%, at least 300%, at least 400%, at least 500%, or at least 1000%.

An “increased” or “enhanced” amount is typically a “statistically significant” amount, and may include an increase that is 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000, 10 000 times or more) (including all integers and decimal points in between and above 1, e.g., 1.5, 1.6, 1.7. 1.8, etc.) an amount or level described herein.

A “decreased,” “inhibiting,” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein. For example, in specific embodiments, germination or colonization associated with Saprolegnia spores in marine or freshwater containing BA is decreased relative to marine or freshwater without containing BA by at least 10%, at least 15%, at least 20%, at least 25%, at least 30%, at least 40%, at least 50%, at least 60%, at least 70%, at least 80%, at least 90%, at least 100%, at least 150%, at least 200%, at least 300%, at least 400%, at least 500%, or at least 1000%.

The terms “marine” or “freshwater” refer to the natural environment of an aquatic animal. The term “marine” refers to any environment relating to the oceans or seas wherein the water is saline. The term “freshwater” refers to, but is not limited to, lakes, ponds, rivers, streams, brooks or any other low salinity water.

By “statistically significant,” it is meant that the result was unlikely to have occurred by chance. Statistical significance can be determined by any method known in the art.

Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95%, 96%, 97%, 98%, 99% or greater of some given quantity.

As used herein, the term “treatment” is defined as the application or administration (e.g., a bath) of a therapeutic agent to a subject (e.g. a fish), or application or administration of the therapeutic agent to an isolated tissue (e.g., fish eggs) or cell line from a patient, who has a disease (e.g., Saprolegnia infections), a symptom of disease or a predisposition toward a disease, with the purpose to cure, heal, alleviate, relieve, alter, remedy, ameliorate, improve or affect the disease, the symptoms of disease, or the predisposition toward disease. For example, “treatment” of a subject in whom no symptoms or clinically relevant manifestations of a disease or disorder have been identified is preventive or prophylactic therapy, whereas clinical, curative, or palliative “treatment” of a subject in whom symptoms or clinically relevant manifestations of a disease or disorder have been identified generally does not constitute preventive or prophylactic therapy.

Treatment of Saprolegnia infections in a fish, for example, includes inhibiting or preventing colonization of infectious stages of Saprolegnia or killing of infectious and/or multiplying stages of Saprolegnia like hyphae. Treating Saprolegnia infections in a fish and preventing Saprolegnia infection progression can include alleviating or preventing symptoms, disorders or clinical disease associated with Saprolegnia infections, thereby curing an infection and restituting the health of the fish or through prophylactic treatment, preventing clinical manifestation of disease to occur. Each form of treatment may be considered a distinct aspect of the disclosure.

The term “microorganism” as used herein refers to any bacteria, fungus, oomycete or Arthropode, for example, Saprolegnia or Sea louse.

The term “chitin-containing microorganism” refers to organisms carrying a coat, membrane, cell wall or exoskeleton containing chitin or chitosan-derived material, for example, Saprolegnia or Sea louse.

The term “chitin-containing microorganism infection” refers to any pathological or non-pathological presence of at least a chitin-containing microorganism on or in an aquatic animal.

The term “chitin-containing microorganism colonization” refers to presence of at least one microorganism carrying chitin (deacetylated chitosan) as part of their cell membrane or their exoskeleton. The colonisation may be an infestation. The colonisation may be chronic, acute, sporadic, seasonal, permanent, temporary, systemic etc.

The term “boric acid” refers to an organic compound with the formula H₃BO₃ (sometimes written B(OH)₃) or any chemical compound containing parts or traces of H₃BO₃.

The term “propionic acid” refers to an organic compound with the formula CH₃CH₂COOH, or any chemical compound containing parts or traces of propionic acid CH₃CH₂COOH.

The term “liquid composition” refers to a composition that is substantially liquid. Concentrations may be referred to in g/L.

The term “solid composition” refers to a composition that is substantially solid. Concentrations may be referred to in g/dm³. The solid composition may be for example in pulverised form or in a form suitable for spraying.

The term “effective concentration” refers to the average concentration of the compound or combination of compounds comprised in the present composition that comes into contact with the animal and is sufficient to induce a response in the pathogen/parasite (chitin-containing microorganism). As contemplated here, the term response may include for example a partial or complete removal, displacement or elimination of the pathogen/parasite from the animal.

### EXPERIMENTAL EXAMPLES

### Example 1

### Boric Acid/Propionic Acid—Effect on Saprolegnia Spore Viability in Biofilms

The effect of treating Saprolegnia-spores with combinations of boric acid and propionic acid was investigated in biofilm preparations since this is one of the most challenging conditions under which effects can be tested.

Saprolegnia spores kept in artificial biofilms (Ali et al. 2014) were subject to different treatments with boric acid, propionic acid and combinations of the two chemicals. Concentrations of boric acid ranged from 1 g/L to 4 g/L, while propionic acid concentrations ranged from 31.4 mM to 125.6 mM. The methods used to form biofilms followed are described in Ali et al. (2014). The methods used were as follows.

### Growing Saprolegnia

Pieces of naturally formed biofilm were incubated in glucose-yeast (GY) broth at 15±1° C. for 2-3 days (Stueland et al., 2005). To inhibit the bacterial growth, chloramphenicol (200 mg ml/L) was added to the medium. The growing hyphae was cut into small pieces and transferred to sterile aquarium water (SAW) for zoospore production. Single spores were isolated using GY agar with antibiotics and incubated at 21±1° C. for 2-5 days (Onions et al., 1981). Procedures were repeated until pure cultures were obtained, and these were stored on autoclaved hemp seeds at 4±1° C. (Stueland et al., 2005).

### Identification of Saprolegnia sp. by Morphology

All strains were identified morphologically (Seymour 1970, Willoughby 1985). Single spore cultures were produced as described above, and small plugs of the growing mycelium was placed in GY broth and incubated for 2-3 days at 15±1° C. Bundles of hyphae formed on the plugs and were washed with SAW. Then incubated with sterile hemp seeds (in SAW) at 3 different temperatures; 5, 15 and 20±1° C. Hemp seeds were assessed for production of oogonia twice weekly by a stereomicroscope, in total over 12-weeks.

### Molecular Identification of Saprolegnia

Saprolegnia isolates obtained from biofilm were then identified by molecular methods as described (Ali et al. 2013). Genomic DNA was extracted from 20 mg mycelia using CTAB miniprep extraction protocol (Gardes and Bruns, 1993). The ITS region was amplified using universal primers ITS1-ITS4 (White et al., 1990) and PCR products were visualized by electrophoresis, and stained with Gelred (Huang et al., 2010). PCR products were sequenced and the contigs were assembled and controlled in BioEdit (Hall, 1999). Sequences were compared to well-annotated Saprolegnia reference strains.

### Zoospore Production and Biofilm Production

Spores were produced to form biofilms. Saprolegnia hyphae were excised from GY agar plates and then incubated in GY broth (15±1° C., 2 days), giving additional hyphal growth. Young hyphae were washed in SAW, transferred to two glass bottles containing one liter of SAW and incubated at 21±1° C. (24 h) to allow zoospore production. Suspensions of zoospores were filtered (tea filter, 0.5 mm pores) resulting in zoospore encystment. Cysts were counted by hemocytometer, the cyst suspensions were adjusted to 1.0±104 spores/L. The suspension was then used in the biofilm formation studies (chambered cover glass system) within 3 h of counting.

Treatment with Boric Acid and Propionic Acid

The biofilm preparations were then subject to treatment with boric acid (BA) at increasing concentrations (1 g/L to 4 g/L), propionic acid (PA; 31.4 mM/L to 125.6 mM/L) or a combination of the two, covering the same concentration range.

Treatment with BA and/or PA was for 4 hours in stagnate, aerated water after which the water circulation was resumed. Biofilm preparations were tested for viability as described (Ali et al. 2014). In brief, CellTiter 96 aqueous one solution cell proliferation assay (Promega) was used. The test is based on tetrazolium compound [3-(4,5-dimethylthiazol-2-yl)-5-(3-carboxy-methoxyphenyl)-2-(4-sulfophenyl)-2H-tetrazolium, inner salt; MTS] being reduced by viable cells resulting in the formation of formazan products from MTS. The reaction is based on mitochondrial enzymes being active (dehydrogenase) (Lopes et al. 2013), and is an indicator of metabolic rate of the mitochondria.

Tests were run in 72-well plates with biofilm formed in the wells. BA and/or PA was then added to each tested well (except controls) at increasing concentrations, 1-4 g/L for BA and 31.4 to 125.6 mM/L for PA. Bronopol was used as a positive treatment but at 300 mg/L concentration, and SAW as negative control. Wells with the MTS reagent without spores were used as a background control. The plates were incubated at 15-20° C. Reading was done following 4 h incubation of BA and PA. Formazan product was measured at 490 nm. MTS reduction is proportional to the number and activity of live spores. Spore viability was calculated by: % viability=100×(OD490 value for the sample−mean background OD490)/(mean OD490 for non-treated water control−mean background OD490).

### Results

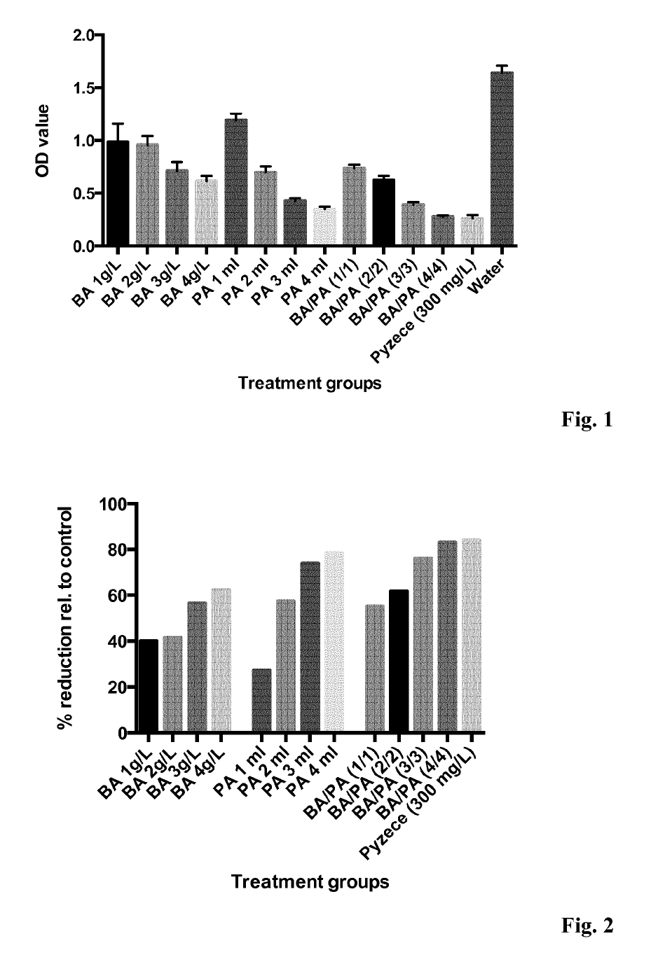

The OD (optical density) values for the different treatment groups are illustrated in FIGS. 1 and 2. Water is the control (non-treated) with an OD value around 1.7. Treatment with BA shows a declining viability from 1 g/L and up to 4 g/L. The findings are similar for PA, with a lower OD value at 4 ml added (equivalent to 125.6 mM/L). Combination of BA and PA gives the lowest viability scores (approx. 0.3). Pyzece at 300 mg/L giimuves the same viability level. It should be added that this concentration of pyceze is 5× what would be a tolerable level for eggs and/or fry of Atlantic salmon. 50 mg/L of pyzece treatment for 1 h results in egg mortality, and standard concentration for prevention of Saprolegniasis in eggs is 30 mg/L. Statistical evaluation shows that PA (4 ml) gives significantly lower viability than 4 g/L of BA. PA/BA (4/4) is not significantly lower than PA (4 ml) but it is significantly lower then BA (4 g/L).

### Example 2

### Safety of Boric Acid/Propionic Acid Treatment in Atlantic Salmon Parr

Atlantic salmon (Salmo salar L.), 4-5 g size (n=30) were subject to treatment with boric acid/propionic acid (BA/PA). The parr were transferred from a holding tank (freshwater, recirculation system at the Norwegian University of Life Sciences/Norwegian Veterinary Institute, wet lab) to a 12 L bucket by dip-netting. The water was aerated throughout the study.

After being held in the water for approximately 5 min, BA and PA were added to the water to give a concentration of 4 mg/L and 125.6 mM, respectively. These solutions were added to the bucket and mixed into the water by gentle stirring. The fish were left for 20 min and observed more or less continuously during this period for change in behaviour. No effect was seen on the fish, they swam around in the bucket and no fish showed any sign of disturbance. No fish died over the 20 min observation period. At the end of the 20 min observation period, an additional dose of BA was added to give a final concentration of 8 mg/L of BA and 251.2 mM of PA. The fish were observed for an additional 20 min. No mortalities occurred.

With the purpose to assess potential impact of pH change as a consequence of adding BA and PA, aquarium water was collected (100 ml), and kept at 15° C. BA and PA was added to the final concentration of 4 mg/L and 125.6 mM, respectively. pH change was minimal from pH of 7.3 in non-added aquarium water to 7.15-7.2 after addition of BA and PA.

These preliminary studies show that BA/PA is safe for use at the recommended concentrations for parr of Atlantic salmon, 4-5 g size.

### Example 3

### In Vivo Studies

| Conditions | Observations |
|---|---|
| BA at 4 mg/L of | Toxic to Atlantic salmon in sea water (32 ppm |
| seawater and | salinity), fish die within 5 minutes after adding |
| PA 52 ppm/L | the solution. |
| BA (4 mg/L) and | Reduced the number of attached sea lice by 66% |
| 11 ppm/L of PA, | by 4 days post treatment and 83% at 8 days post |
| 5 minutes exposure | treatment. |
| BA (1 mg/L) and | Reduced the number of attached sea lice by 40% |
| 2.75 ppm/L of PA, | by 4 days post treatment. |
| 10 min exposure | |
| 2.75 ppm/L of PA, | Reduced the number of attached sea lice by 30% |
| 15 min exposure | by 4 days post treatment. |

Water salinity: 32 ppm; Temperature: 8.5° C.; Oxygen: minimum 8 mg/L in water during treatment (8-10 mg/L measured).

### Example 4

### In Vitro Studies

| Conditions | Observations |
|---|---|
| Adult sea lice (with egg strings) and chalimus | No moving stages |
| were exposed to BA (4 mg/ml) and 2.75 ppm | were observed for |
| PA for 30 minutes. They were then transferred | 2 hours. |
| to normal seawater (32 ppm) and observed for 2 | |
| hours. | |
| Adult sea lice (with egg strings) and chalimus | No moving lice were |
| were exposed to BA (4 mg/ml) and 5.5 ppm PA | observed after 2 hours. |
| for 60 minutes. | |
| Adult sea lice (with egg strings) and chalimus | No toxic effect was |
| were exposed to PA only at 1.38 ppm for 1 h. | observed (still |
| | movable stages). |
| Control groups were exposed to sea water. | Movement was still |
| | seen 48 h post transfer |
| | to in vitro conditions. |

### REFERENCES

1. Ali, S. E.; Thoen, E.; Evensen, O.; Skaar, I. Boric acid inhibits germination and colonization of saprolegnia spores in vitro and in vivo. PloS one 2014, 9, e91878.
2. Stueland, S.; Hatai, K.; Skaar, I. Morphological and physiological characteristics of saprolegnia spp. Strains pathogenic to atlantic salmon, salmo salar 1. Journal offish diseases 2005, 28, 445-453.
3. Onions, A. H. S.; Allsopp, D.; Eggins, H. O. W. Smith's introduction to industrial mycology. 7th ed.; London, 1981.
4. Seymour, R. L. The Genus Saprolegnia. Nova Hedwigia (Beih) 1970, 1-124.
5. Willoughby, L. G. An ecological study of water as the medium for growth and reproduction of the saprolegnia from salmonid fish. Transactions of British Mycological Society 1986, 87.
6. Gardes, M.; Bruns, T. D. Its primers with enhanced specificity for basidiomycetes-application to the identification of mycorrhizae and rusts. Molecular Ecology 1993, 2, 113-118.
7. White, T. J.; Bruns, T.; Lee, S.; Taylor, J. Amplification and direct sequencing of fungal ribosomal ma genes for phylogenetics. Pcr protocols: A guide to methods and applications. 1990.
8. Hall, T. A. Bioedit: A user friendly biological sequence alignment editor and analysis program for windows 95/98/nt. Nucleic Acid Symposium Series 1999, 41, 95-98.
9. Huang, T.-S., Cerenius, L., Söderhäll, K. Analysis of genetic diversity in the crayfish plague fungus, Aphanomyces astaci, by random amplification of polymorphic DNA. Aquaculture 1994, 126, 1-10.
10. Lopes G, Pinto E, Andrade P B, Valentao P Antifungal activity of phlorotannins against dermatophytes and yeasts: approaches to the mechanism of action and influence on Candida albicans virulence factor. PLoS One, 2013, 8: e72203.

1. A composition for the treatment of sea louse infection or colonization of an aquatic animal, the composition comprising a mineral acid and a carboxylic acid.

2. The composition according to claim 1, wherein the mineral acid is boric acid.

3. The composition according to claim 1, wherein the carboxylic acid is propionic acid.

4. The composition according to claim 1, wherein the composition has a concentration of the mineral acid not less than about 0.2 g/L and a concentration of carboxylic acid not less than about 0.2 g/L.

5.-6. (canceled)

7. The composition of claim 1 further comprising one or more stabilisers, additives or excipients.

8. (canceled)

9. The composition according to claim 1, wherein the aquatic animal is a fish selected from the group consisting of: a fish egg, a Juvenile fish, a fry, a fingerling, an adult fish, and an off-spring of the fish.

10. The composition according to claim 9, wherein the aquatic animal is a fish selected from the group consisting of: a brown trout, an Atlantic salmon, a rainbow trout, a coho salmon, a catfish, a pike, an arctic char, an eel, a roach, a carp, a sturgeon, a kissing gourami, a guppy, a swordfish, a tilapia, a cod and a platyfish.

11. The composition according to claim 1, wherein the aquatic animal is selected from the group consisting: an aquatic mammal, an aquatic bird, an aquatic reptile, an amphibian, and an aquatic invertebrate.

12.-20. (canceled)

21. A method for treating sea louse infection or colonization in an aquatic animal, the method comprising contacting the aquatic animal with the composition of claim 1.

22.-23. (canceled)

24. A kit for administering the composition of claim 1 to an aquatic animal to treat sea louse infection or colonization, the kit comprising: a container having the composition, the composition dissolvable in a carrier to provide an anti-sea louse solution; and packaging that includes an instruction directing contacting the aquatic animal with the anti-sea louse solution to treat the sea louse infection or colonization.

25.-29. (canceled)

30. The method of claim 21, wherein the animal is contacted or directed to be contacted with the composition for a period of time of between about 24 hours and about 96 hours.

31. The method of claim 21, wherein the animal is contacted or directed to be contacted with the mineral acid and the carboxylic acid sequentially, concomitantly or simultaneous.
